(19) United States
(12) Patent Application Publication
(10) Pub. No.: US 2007/0212434 A1
(43) Pub. Date: Sep. 13, 2007

(54) Compositions and methods for human use containing fulvic acid

(76) Inventors: Kenneth S. Day, Modesto, CA (US); George A. Hansen, Salt Lake City, UT (US)

Correspondence Address: TRASK BRITT, P.O. BOX 2550, SALT LAKE CITY, UT, 84110, US

(21) Appl. No.: 11/369,655

(22) Filed: Mar. 7, 2006

(51) Int. Cl. A61K 36/10

(52) U.S. Cl. 424/762

(57) ABSTRACT

The present invention provides compositions comprising fulvic acid. The present invention further provides methods and compositions for promoting hair growth involving the use of fulvic acid.

### TECHNICAL FIELD

This invention relates to the field of cosmetics and nutritional supplements generally and, more specifically, to methods and compositions involving the use of fulvic acid.

### BACKGROUND

Fulvic acids are natural compounds that can be found, among other places, in soils, rivers, lakes and ocean sediments. They are a family of organic acids that come from soil humus. Plants absorb small quantities of fulvic acids from the soil as a normal part of their way of accessing mineral nutrients like iron, zinc, manganese and copper (Chen and Aviad, 1990). Research confirms that plants retain small amounts of fulvic acids (Ghabbour and Davies, 1994) after they have been taken up from the soil. Once absorbed, fulvic acids stimulate the metabolic activity of plants in a number of ways (Nardi, 1996).

In undisturbed natural settings, the soils are rich in fulvic acids. Animals and people eat the plants containing them and thereby benefit from them as a normal part of their diet.

In modern times, the carbon cycle has been interrupted to varying degrees on most large-scale farms. Farms depend on chemical fertilizers and pesticides, burn crop stubble, and till the soil intensely (rather than depend on crop rotation and manures or composts). Practical and economic considerations have driven the vast majority of farms in the developed world to adopt such practices. These farming methods have been shown to dramatically reduce the amount and quality of humus and other organic matter in the soil. Under intense cultivation, soils may lose half or more of their active humus fractions in three to five years (Freeman, 1969). Numerous studies show that fulvic acids are consumed much faster than the other constituents of soil humus. One study showed that soil from forest lands in Poland, for example, lost over 90% of its soluble fulvic acids from plant uptake and microbial degradation after being cleared and replaced with wheat and corn farms (Insam, 1996). Other soil fractions were less impacted.

A general reduction in fulvic acids in agricultural soils logically results in a lower intake of fulvic acids in the diet of the average person. Furthermore, much of the fulvic acid that does make it into our diet may have been degraded by heat during processing.

Scientists are becoming increasingly aware of the importance of organic soil amendments in general, and of fulvic and humic acids in particular (Piccolo, 1996). Producers of food and fiber are using commercial concentrates of fulvic acids (and humic acids) to an ever increasing extent. Fulvic and humic acid containing compositions promote better plant growth and improve the uptake of fertilizers and mineral nutrients into plants (Day, 2000). The effects of fulvic acids on plant growth have been studied and documented in great detail (Chen, 1999). Extensive scientific literature reviews are available on the subject (Vaughan and Malcolm, 1985, Chen and Aviad, 1990 and Nardi, 1996).

As the active ingredient in peat based mud baths, fulvic acids have been used and studied (Grassi, 2003) for many years in the treatment of various ailments by topical contact with the skin (Beer et al., 2003). Scientific documentation of these benefits is becoming more extensive. In recent years, there has also been a dramatic rise in the use of fulvic acid fortified mineral supplements (UC Berkley Wellness Letter, June 1997). Studies to document the benefits of fulvic acids in the diet are beginning to appear in international scientific publications (Madej, 1993). There has also been a rise in the use and study of fulvic acids as supplements in animal feeds (Covington et al., 1997).

The growing body of scientific literature from various domains sheds a great deal of light on potential uses for fulvic acids in human nutrition, in topical applications to the skin, and for the treatment of various diseases. Although much research remains to be done on the subject, early indications are quite favorable to support the supplementation of fulvic acids in the diet and for their use in topical skin treatments.

TABLE 1
A Non-Comprehensive List of Documented Benefits of Fulvic Acid

| Number | Use | Organ or System | Benefit | Reference Source |
|---|---|---|---|---|
| 1 | External | Skin | Improved Absorption of Minerals | Beer, 2000 |
| 2 | Internal | Cellular & GI Tract | Detoxification of Environmental Poisons | Lahtosh, 1991 |
| 3 | Internal | Auto-immune | Raised white blood cell count & activity | Baj, 1993 |
| 4 | Internal | Auto-Immune | Improved Immune function in HIV positive persons | Dekker, 2001 |
| 5 | Internal | Auto-Immune | Recovery from chronic fatigue due to Candida | Dekker, 2001 |
| 6 | Internal | GI Tract | Faster healing of gingivitis and other gum diseases | Tolpa, 1998 |
| 7 | Internal | Muscles | Relief from fibromyalgia | Dekker, 2001 |
| 8 | Internal | Cellular | Faster respiration rates for improved metabolism | Visser, 1987 |
| 9 | External | Skin | Relaxation of smooth muscles under the skin | Beer, 2000 |
| 10 | External | Skin | Faster healing from various infections | Dekker, 2001 |
| 11 | Internal | Kidneys | Prevention of kidney stones | Fraioli, 2001 |
| 12 | Internal | Cardiopulmonary | Preventative and curative for diabetes | Ghosal, 2003 |
| 13 | Internal | Cardiopulmonary | Reduces hypoglycemia | Bhattacharya (1995) |
| 14 | External | Skin | Enhances skin softness | Beer, 2003 |
| 15 | Internal | Circulatory | Reduced cholesterol and blood glucose | Banaszkiewicz, 1994 |

As there has been substantial benefit attributed to the human use of fulvic acid, a need exists in the art for compositions that contain substantial amounts of these compounds.

### DISCLOSURE OF INVENTION

The present invention encompasses the use of fulvic acid in compositions for human use.

One aspect of the invention relates to compositions for human use having more than 700 parts-per-million (mg/L) of fulvic acid. Exemplary forms of these compositions include, but are not limited to, tablets, capsules, granules, supplements, foods, creams, ointments, liquids, emulsions, suspensions, drinks, beverages, sprays, inhalers, suppositories, drops, balms, patches, cosmetics, or other forms designed to deliver fulvic acid to a subject. Preferably the compositions contain between 700 and 20,000 mg/L of fulvic acid.

A further aspect of the present invention relates to compositions for human use having more than 700 mg/L of purified or substantially purified fulvic acid. Exemplary forms of these compositions include, but are not limited to, supplements, foods, creams, ointments, liquids, drinks, beverages, cosmetics, or other forms designed to deliver purified or substantially purified fulvic acid to a subject. Preferably the compositions contain between 700 and 20,000 mg/L of purified substantially purified fulvic acid.

In an additional aspect of the present invention, the fulvic acid is purified or substantially purified from an aquatic source.

In a further aspect of the present invention, the fulvic acid is not purified from coal, shilajit, or peat.

In an additional aspect of the present invention, the fulvic acid is not oxifulvic acid.

The present invention further encompasses methods of restoring hair growth in a subject in need thereof via treatment with a composition containing fulvic acid.

Another aspect of the invention relates to methods of promoting hair growth in a subject comprising delivering to the subject at least 5 mg of fulvic acid per day. More preferably, the subject is delivered 20 to 30 mg of fulvic acid per day. The method of delivery of the fulvic acid can be in any form designed to deliver fulvic acid to a subject such as, but not limited to, supplements, foods, creams, ointments, liquids, drinks, beverages, sprays, inhalers, suppositories, eye drops, and cosmetics. Preferably, the fulvic acid is isolated from an aquatic source.

### MODES FOR CARRYING OUT THE INVENTION

Fulvic acid is a family of organic acids. The term “fulvic acid” is understood by those of skill in the art, as exemplified by U.S. Pat. Nos. 6,874,277, 6,596,900, 6,478,946, and 5,204,368, the contents of the entirety of each of which are hereby incorporated herein by reference. Although fulvic acid has been studied extensively in the fields of geochemistry, agriculture, and environmental chemistry, it is still not widely known by scientists in the medical and nutritional fields. Fulvic acid is not one specific compound. Rather, it is a large family of related organic compounds. The term, fulvic acid, is similar to the terms amino acids or tannic acids. There are 20 different acids known as amino acids that share similar chemical structures, characteristics and biological functions. Tannic acids are polyphenols derived from plants. There are probably hundreds if not thousands of different compounds in the family of tannic acids. Fulvic acids are quite the same in principal. No one knows exactly how many compounds exist within this family of humus derived compounds. In a recent publication, one researcher isolated 4,000 distinct compounds in just one sample of Suwanee River fulvic acid. In addition, fulvic acids derived from aquatic sources are distinct from those isolated from coals or soils (Stevenson, 1994). There can be little doubt, that there are at least several thousand different compounds that are included in the family of fulvic acid.

Fulvic acids, as referred to herein, are the organic acids fraction that is soluble in water under all pH conditions and is in general lower in molecular size and weight and lower in color intensity than humic acids. Fulvic acids are yellow to orange in color when dissolved in water at concentrations of 50 to 500 milligrams per liter. Further, fulvic acids may be defined as the class of compounds which are purified by the standard methods promulgated by the International Humic Substances Society (IHSS) and as outlined in Examples 1 and 2 below.

According to certain embodiments of the present invention, the fulvic acid may be purified or substantially purified before incorporation into a composition for human use or before use in a method to promote hair growth. In certain embodiments, the fulvic acid may be that obtained via purification using XAD-8. In other embodiments of the present invention, the fulvic acid is isolated from an aquatic source. In further embodiments of the present invention, the fulvic acid is not isolated from peat, shilajit, or coal. Further, the fulvic acid may be fulvic acid other than oxifulvic acid.

According to one embodiment of the present invention, a composition for human use containing fulvic acid is provided. The composition comprises at least 700 mg/L fulvic acid and preferably between 700 and 20,000 mg/L fulvic acid. Exemplary forms of these compositions include, but are not limited to, tablets, capsules, granules, supplements, foods, creams, ointments, emulsions, suspensions, liquids, drinks, beverages, sprays, inhalers, suppositories, drops, cosmetics, or other forms designed to deliver fulvic acid to a subject.

The composition for human use may be in the form of a cosmetic. Such cosmetics are exemplified by, but not limited to, lotions, milky lotions, creams, facial packs, ointments, tooth pastes, bathing agents, bath detergents, facial cleansing agents, hair lotions, hair-care compositions, or shampooing agents. Cosmetics of the present invention may be prepared in accordance with a conventional method, and those usually used in cosmetics such as hydrocarbons, waxes, fats and oils, esters, higher fatty acids, higher alcohols, surfactants, perfume, pigments, anticorrosive agents, antioxidants, ultraviolet absorbents, alcohols, pH adjustment agents, various ingredients with medicinal effect can be properly selected and formulated. In addition, such cosmetics may comprise a component having skin cosmeticizing action such as, but not limited to, retinoic acid, α-hydroxy acid, retinol, glycerol, polyethylene glycol, potassium hydroxide, triethanolamine, and other saccharides. The cosmetic may further comprise a component having a hair restoring action including, but not limited to, minoxidil, calpronium chloride, heparin analogs, glyceryl monolinolate, linoleic acid, various crude drug extracts, and the like. The cosmetics of the present invention may be prepared by comprising, adding and/or diluting a composition containing fulvic acid so as to achieve a final concentration of at least 700 mg/L fulvic acid.

In addition, one embodiment of the present invention relates to foods, beverages, or drinks. The foods, beverages, or drinks may be prepared by comprising, adding and/or diluting a composition containing fulvic acid so as to achieve a final dosage concentration of at least 700 mg/L fulvic acid. Methods for preparing foods, beverages, or drinks containing fulvic acid are not particularly limited. For example, manufacturing processes may include, but are not limited to, cooking and processes carried out in accordance with those methods generally employed for foods, beverages, or drinks. In addition, the form of the foods, beverages, or drinks of the present invention are not particularly limited. For example, foods, beverages, or drinks according to the present invention include, but are not limited to, processed agricultural and forest compositions, processed stock raising compositions, processed marine compositions and the like, including processed grain compositions such as processed wheat compositions, processed starch compositions, processed premix compositions, noodles, macaronis, bread, bean jam, buckwheat noodles, wheat-gluten bread, rice noodle, fen-tiao, and packed rice cake; processed fat and oil compositions such as plastic fat and oil, tempura oil, salad oil, mayonnaise, and dressing; processed soybean compositions such as tofu compositions, soybean paste, and fermented soybeans; processed meat compositions such as ham, bacon, pressed ham, and sausage; marine compositions such as frozen ground fish, boiled fish paste, tubular roll of boiled fish paste, cake of ground fish, deep-fried patty of fish paste, fish ball, sinew, fish meat ham and sausage, dried bonito, compositions of processed fish egg, marine cans, and preserved food boiled down in soy sauce (tsukudani); milk compositions such as raw material milk, cream, yogurt, butter, cheese, condensed milk, powder milk, and ice cream; processed vegetable and fruit compositions such as paste, jam, pickled vegetables, fruit beverages, vegetable beverages, and mixed beverages; confectioneries such as chocolates, biscuits, sweet bun, cake, rice cake snacks, and rice snacks; alcohol beverages such as sake, Chinese liquor, wine, whisky, Japanese distilled liquor (shochu), vodka, brandy, gin, rum, beer, refreshing alcoholic beverages, fruit liquor, and liqueur; luxury drinks such as green tea, tea, oolong tea, coffee, soft drinks and lacetic acid drinks; seasonings such as soy sauce, sauce, vinegar, and sweet rice wine; canned, binned or pouched foods such as rice topped cooked beef and vegetable, rice boiled together with meat and vegetables in a small pot, steamed rice with red beans, curry roux and rice, and other precooked foods; semi-dry or concentrated foods such as liver pastes and other spreads, soups for buckwheat noodles or wheat noodles, and concentrated soups; dry foods such as instant noodles, instant curry roux, instant coffee, powder juice, powder soup, instant soybean paste (miso) soup, precooked foods, precooked beverages, and precooked soup; frozen foods such as sukiyaki, pot-steamed hotchpotch, split and grilled eel, hamburger steak, shao-mai, dumpling stuffed with minced pork, various sticks, and fruit cocktails; solid foods; liquid foods (soups or the like); spices; and the like.

In further embodiments, the fulvic acid may be mixed with one or more carriers, adjuvants, and/or diluents to form a composition for human use. See, e.g., Remington's Pharmaceutical Science 18th Ed. (1990, Mack Publishing Co., Easton, Pa.), Goodman and Gilman's

The Pharmacologic Basis of Therapeutics 10th Ed. (2001, McGraw-Hill Professional). The fulvic acid, with or without an adjuvant and/or a carrier, may be administered to a subject in a manner that will provide the fulvic acid to the subject. Examples include, but are not limited to, site-specific injection, systemic injection, and/or administration intravenously, orally, and/or topically. Compositions containing fulvic acid may be shaped into tablets, granules, capsules, emulsions, suspensions, or the like which may be taken orally. Further, the compositions containing fulvic acid may use alternative delivery devices and methods such as, but not limited to, sprays, inhalers, suppositories, and drops.

Other embodiments of the invention relate to methods of promoting hair growth in a subject comprising delivering to the subject at least 5 mg of fulvic acid per day. More preferably, from 20 to 30 mg of fulvic acid are delivered to the subject per day. Methods of delivering the fulvic acid to a subject include, but are not limited to, injection, tablets, capsules, granules, supplements, foods, creams, ointments, suspensions, emulsions, liquids, drinks, beverages, sprays, inhalers, suppositories, eye drops, and cosmetics.

The present invention is further described in the following examples, which are offered by way of illustration and are not intended to limit the invention in any manner.

### EXAMPLES

### Example 1

Isolation of Fulvic Acids from Soil

A number of methods for the extraction of humic substances from soil using sodium hydroxide solution have been published. These methods are generally successful and yield comparable results. The following is a method which has been developed by the International Humic Substance Society (IHSS) as an acceptable method for the extraction of humic substances from soils. An important component of this method is the use of an adsorbent resin in the purification process.

XAD-8 is a nonionic, macroporous (pore size 25 μm), methyl methacrylate ester resin (see “Fractionation of Humic Substances Adsorption”). Because it is sometimes difficult to obtain, it may be necessary to use an alternative resin such as Polyclar, which is a cross-linked poly(vinylpyrrolidone) (PVP) (De Nobili et al., 1990; Watanabe & Kuwatsuka, 1991) or other equivalent resin. As an alternative to XAD-8, the DAX-8 resin may also be used.

Purification Protocol:

Remove roots and sieve the dried soil sample to pass a 2.0-mm sieve. Equilibrate the sample to a pH value between 1 to 2 with 1 M HCl at room temperature. Adjust the solution volume with 0.1 M HCl to provide a final concentration that has a ratio of 10 mL liquid/1 g dry sample. Shake the suspension for one hour and then separate the supernatant from the residue by decantation after allowing the solution to settle or by low speed centrifugation. Save the supernatant (FA Extract 1) for the isolation of fulvic acid using XAD-8 resin (Rohm & Haas Co., Philadelphia, Pa.).

Neutralize the soil residue with 1 M NaOH to pH=7.0 then add 0.1 M NaOH under an atmosphere of N₂ to give a final extractant to soil ratio of 10:1. Extract the suspension under N₂ with intermittent shaking for a minimum of four hours. Allow the alkaline suspension to settle overnight and collect the supernatant by means of decantation or centrifugation. Acidify the supernatant with 6 M HCl with constant stirring to pH=1.0 and then allow the suspension to stand for 12 to 16 hours. Centrifuge to separate the humic acid (precipitate) and fulvic acid (supernatant-FA Extract 2) fractions.

Redissolve the humic acid fraction by adding a minimum volume of 0.1 M KOH under N₂. Add solid KCl to attain a concentration of 0.3 M (K+) and then centrifuge at high speed to remove the suspended solids. Reprecipitate the humic acid by adding 6 M HCl with constant stirring to pH=1.0 and allow the suspension to stand again for 12 to 16 hours. Centrifuge and discard the supernatant. Suspend the humic acid precipitate in 0.1 M HCl/0.3 M HF solution in a plastic container and shake overnight at room temperature. Centrifuge and repeat the HCl/HF treatment, if necessary, until the ash content is below 1%. Transfer the precipitate to a Visking dialysis tube by slurrying with water and dialyze against distilled water until the dialysis water gives a negative Cl-test with silver nitrate AgNO₃. Freeze dry the humic acid.

Pass the supernatant designated “FA Extract 1” through a column of XAD-8 (0.15 mL of resin per gram of initial sample dry weight at a flow rate of 15 bed volumes per hour). Discard the effluent, rinse the XAD-8 column containing sorbed fulvic acid with 0.65 column volumes of distilled H₂O. Back elute the XAD-8 column with 1 column volume of 0.1 M NaOH, followed by 2 to 3 column volumes of distilled H₂O. Immediately acidify the solution with 6 M HCl to pH=1.0. Add concentrated HF to a final concentration of 0.3 M HF. The solution volume should be sufficient to maintain the fulvic acid in solution.

Pass the supernatant designated “FA Extract 2” through a column of XAD-8 (1.0 mL of resin per gram of initial sample dry weight). Repeat the back elution and acidification as for “FA Extract 1” above. Combine the final eluates from each of the fulvic acid extracts and pass this solution through XAD-8 resin in a glass column (column volume should be one-fifth of sample volume). Rinse with 0.65 column volumes of distilled H₂O. Back elute with 1 column volume of 0.1 M NaOH followed by two column volumes of distilled H₂O. Pass the eluate through H+-saturated cation exchange resin (Bio-Rad AG-MP-5 (Bio-Rad, Richmond, Calif.) using three times the mole of Na ions in solution). Freeze dry the eluate to recover the H+-saturated fulvic acid.

### Example 2

Isolation of Fulvic Acids from Aqueous Solution

This protocol is adapted from: Aiken, G. R. (1985) “Isolation and concentration techniques for aquatic humic substances,” in G. R. Aiken, D. M. McKnight, R. L. Wershaw, and P. MacCarthy (Eds.), Humic substances in soil, sediment and water: geochemistry and isolation. Wiley-Interscience, New York, and is republished by the IHSS as a standard method of isolating humic and fulvic acids.

Purification Protocol:

Filter water with a 0.45 μm silver or polymer membrane filter. Lower pH to 2.0 with HCl. Pass sample through column of XAD-8 resin to retain humic and fulvic acids. The preparative cleaning of the resin is described by Thurman and Malcolm (1981). Elute HA and FA from the column with 0.1 M NaOH, in the reverse direction. Acidify immediately with HCl to avoid oxidation of humic substances. Re-concentrate on a smaller XAD-8 column. Elute with NaOH and acidify. The eluted DOC should contain more that 500 mg C/L.

Adjust pH to 1.0 with HCl. Centrifuge to separate the humic acid (HA) from the fulvic acid (FA) fraction. Wash HA with water until wash is negative to the AgNO₃ test for chloride. Add sufficient 0.1 M NaOH to dissolve HA and then acidify by passing through a strong acid resin column.

Adjust FA fraction to pH 2.0 with NaOH and re-adsorb FA fraction on XAD-8. Wash with one void volume of distilled water to remove the salt. Reverse flow and elute column with 0.1 M NaOH. Immediately pass FA eluate through cation-exchange resin and hydrogen saturate. Pass HA in 0.1 M NaOH through cation-exchange resin and hydrogen saturate. Repeat until Na⁺ is less than 1 mg/L.

The Fulvic acid content of a sample can be determined by the following procedure. Remove the resin from a XAD-8 column, weigh the resin, and repack the column with the resin. Pass an aliquot of purified fulvic acid obtained by the above procedure over the XAD-8 in the column. Remove the XAD-8 from the column and centrifuge at low speed to remove the excess water from the resin. Weight the resin and bound fulvic acid. The weight of fulvic acid in the aliquot is roughly equal to the increased weight of the resin.

### Example 3

Fulvic acid is used for preparing gel and ointment compositions, containing also herb extracts synergistically improving the therapeutic effect with respect to certain diseases. For example, a gel and ointment against varicose ulcer of the shank is prepared as follows: 20 g of hippocastanaceous extract, 10 g of calendula extract, 60 g of glycerol, 0.1 g of salicylic acid, 1.0 g of distilled water, 8.8 g of Aerosil (R)) (colloidal silica), and enough fulvic acid to achieve a final concentration of 1000 mg/L are used in order to obtain a gel form of the preparation.

Liquid (non-volatile) ingredients are sterilized before use, by means of heating under reflux for two hours. Herb extracts are combined with glycerol and an aqueous solution fulvic acid and also with menthol, and silica is gradually added to the obtained mixture, under continuous stirring.

Similarly, in order to obtain an ointment composition, the following ingredients are used: 20 g of hippocastanaceous extract, 10 g of calendula extract, 0.1 g of salicylic acid, 2.0 g of Aerosil (R) (colloidal silica), and enough fulvic acid to achieve a final concentration of 1000 mg/L.

As fatty components, a mixture of the following substances is used: 22 g of eucerine and 45.8 g of petrolatum. Herb extracts are sterilized by heating under reflux for approximately two hours. Eucerine and petrolatum are similarity sterilized. Liquid ingredients are carefully combined with silica to obtain a gel, which in turn is triturated with sterilized and fatty components cooled down to room temperature. A stable ointment is obtained which does not separate when stored.

The gel and ointment obtained above may be simultaneously applied in the treatment of varicose ulcer of the shank. Ulcers are treated with the gel preparation while the surrounding, nonaffected skin is treated with ointment. Addition of colloidal silica is believed to be responsible for prompt dessication while the herbal and fulvic acid ingredients are believed to be responsible for the curing effect of the preparation. Fatty components help to keep elastic the crust and the skin. The results obtained are compared with a control group of patients treated in a classic way. Those who received the new treatment are selected from a group of subjects suffering from the disease for many months (sometimes years) without noticeable positive effects. Subjects treated with compositions according to the invention show better results than control patients.

### Example 4

Fulvic acid is used to prepare pharmaceutical formulations in the form of tablets, or material to be placed in capsules.

A sterile peat-derived bioactive composition in powdered form is combined with a carrier in a weight ratio of 1:9. As a carrier, MYVATEX (R)TL (tradename of Eastman-Kodak), a mixture of lactose and lubricating substances, is used in a weight ratio of 44:1. Lactose of 50 mesh particle size and MYVATEX (R)TL are finely disintegrated so that approximately 70% of its mass is passed through a 100 mesh screen. A part of the resulting mixture of active composition and carrier is formulated into tablets containing 1 mg of fulvic acid. The total mass of each tablet is 50 mg. The other part of the same mixture of active composition and a carrier is granulated using q.s. of ethanol (40% by volume). Granules are sieved and ground if necessary and then filled in capsules in such a quantity that each capsule contained 1 mg of fulvic acid.

The tablets obtained as above are tested in order to measure the time of their disintegration in an artificial gastric juice at 37° C.+/−2° C. using Erweka equipment. The artificial gastric juice is prepared as follows: 2.0 g of sodium chloride and 3.2 g of pepsin are dissolved in 7 ml of hydrochloric acid and distilled water is added up to a total volume of 700 ml. The pH-value of the resulting solution is approximately 1.2. Disintegration time of a tablet, having a diameter of 5.1 mm and a total mass of 0.0498 g, is determined.

Further examples relate to numerous cosmetic preparations according to the present invention, having different forms composition and being designed for different applications, containing the beneficial addition of fulvic acid. Among others, preparations such as tonics, balms, creams, milks, shampoos, foaming bath compositions etc. are described.

### Example 5

A reaction vessel equipped with a stirrer is charged with 150 g of camomile extract obtained by the extraction of camomile inflorescence with a 1:1 ethanol:water solution, as well as enough fulvic acid to achieve a final concentration of 1000 mg/L. 50 g of glycerol are added to the mixture obtained. The three substances are stirred to obtain a uniform mixture. Subsequently, a second mixture as previously formulated is introduced into the same vessel. It comprises 340 g of a 95:5 ethanol:water solution, 1 g of salicylic acid and 0.5 g of menthol. The two mixtures are combined by stirring to form a uniform solution. Next, 3 g of a fragrant composition TILIANA H4308 are added. The solution is then brought to a total volume of 700 ml by adding 454.5 g of distilled water; stirring is continued until a homogeneous mixture is obtained.

In the above procedure, 86% glycerol, menthol and water, and ethanol in a concentration of 95% are used.

The tonic preparation obtained above is suitable for all kinds of skin.

### Example 6

The procedure described in Example 5 above is repeated, the only difference being that instead of camomile extract and the TILIANA H4308 fragrant composition, a marigold flowers extract and a composition FINUS H4625 are used in the same way and the same molar and volume ratios. The resulting tonic preparation is suitable for dry and fragile skin.

### Example 7

The procedure of Example 5 is repeated, except that an extract of sage leaves is chosen instead of camomile extract, and the fragrance LELIA 90368 (Pollena-Aroma, Warsaw) is chosen instead of TILLANA H4308. The extract of sage leaves is obtained by extracting dried sage leaves with ethanol at 50° C. and has a brownish color, and a characteristic sage odor. The resulting face care agent is suitable for greasy skin.

### Example 8

The following composition is a gel for avoiding or treating periodontosis: 24 g chamomile extract, 3 g sage leaf extract, 0.3 g salicylic acid, 0.2 g methanol, 100 g of a commercial gel base, and enough fulvic acid to achieve a final concentration of 1000 mg/L.

### Example 9

The following components are introduced into a reaction vessel of a volume of 2000 ml, equipped with a mechanical stirrer:

270 g of camomile extract obtainable by extraction of camomile inflorescence with 50% ethanol; the extract was a red-brown liquid, having a density of 0.9160-0.9503 g/ml and an ethanol content of approximately 55% by volume;

50 g of glycerol;

30 g of a saponaria officinalis extract obtainable by extracting saponaria officinalis roots with 70% ethanol; the extract was a red-brown liquid, the density was 0.9630-0.9810 g/ml, and the ethanol content approximately 75% by volume; and

Enough fulvic acid to achieve a final concentration of 1000 mg/L.

The ingredients listed above are mixed thoroughly. A previously prepared solution of 1 g of salicylic acid in 260 g of 95% ethanol is added thereto. To the combined solution, 383 g of distilled water and 5 g of fragrant composition TILIANA H3408 are added and stirred until a uniform solution is obtained. The resulting preparation is suitable as a hair care preparation.

### Example 10

The procedure described in Example 9 is followed except that instead of camomile extract and TILIANA H4308 composition there are used in the same sequence and ratio: horsetail herb extract and the fragrant composition FINUS H 4625. Horsetail herb extract is a green-brown liquid of a density of 0.9160-0.9503 g/ml and an ethanol content of 55% by volume. The resulting preparation is suitable for all kinds of hair.

### Example 11

The procedure as described in Example 9 is repeated. The only difference is that, instead of camomile extract and TILIANA H4308 composition, stinging nettle leaves extract and fragrant composition LELIA 90368 in the same sequence and ratio are used.

### Example 12

In general, cosmetic milks are dispersions of fatty substances acting in both chemical and mechanical ways on the skin. In fact, due to a convenient way of application and better interaction of the fluid and the skin, it is very appropriate to use liquid, more specifically emulsion creams. They can easily penetrate to deeper layers of the skin and thus prevent changes of the skin due to age. Cosmetic milks are used mainly to clean a dry and fragile skin. Accordingly, they must not contain any aggressive volatile oils, while frequently they contain suitable herb extracts like camomile extract or wheat germ extract. Addition of fulvic acid to such cosmetic milks further improves their positive effects. An exemplary recipe is as follows: 20 g aloe extract, 3 g glycerol, 2 g eucerine, 1 g white paraffin oil, 1 g triethylamine, 4 g Aerosil® (colloid silica), and enough fulvic acid to achieve a final concentration of 1000 mg/L.

### Example 13

Fulvic acid and selected fatty carriers are used in a classic nourishing and regenerative cream formula. Fulvic acid is used in an amount to obtain a final concentration of 1000 to 20,000 mg/L in combination with a herb extract (selection depends on the type of skin for which the cream is intended) in an amount of at least 0.05-1.00% by weight, antibacterial preparation in an amount of 0.05-1.00% by weight, synthetic fragrant composition in an amount of 0.01-0.05% by weight and a fatty carrier in the form of a water emulsion, constituting 97.00-99.50% by weight of the whole composition. The fatty composition needs to be a good carrier for the active ingredients and to be well accepted by the skin. Preferably, it is an emulsion of (all amounts in % by weight) 35-45 eucerine, 8-14 petrolatum, 2.5-4 olive oil, 6-10 glycerol and 35-40 water. Preferred herb extracts are marigold flower extract, camomile extract, thyme extract and the like.

### Example 14

An after-shave preparation containing fulvic acid in an amount to obtain a final concentration of 1,000 to 20,000 mg/L, herb extracts in an amount of 1-30% by weight, glycerol in an amount of 1-8% by weight, salicylic acid and menthol in aqueous-alcohol solution is prepared. Preferred herb extracts are: camomile, marigold, thyme, aloe extract and similar beneficial herb extracts. Addition of glycerol is also beneficial due to its influence on the elasticity of the skin. It speeds up the spreading of the preparation on the face as well as the penetration into the deeper layers of the skin, thus enhancing the beneficial effects of the fulvic acid and herb extracts.

### Example 15

A shampoo composition is prepared according to the following recipe: fulvic acid in an amount to obtain a final concentration of 1,000 to 20,000 mg/L, 15 g fuller's herb extract, 20 g stinging nettle leaves extract, 30 g GAMAL SBS-11 (detergent), 20 g GAMAL NO-3 (detergent), 0.4 g aseptina, 1.6 g ethanol, 0.04 g BRONOPOL (preservative), 6 g sodium chloride, 106 g of water.

An alternative shampoo composition is prepared according to the following recipe: fulvic acid in an amount to obtain a final concentration of 1,000 to 20,000 mg/L, 13 g horse chestnut extract, 22 g marigold extract, 30 g GAMAL SBS-11 (detergent), 20 g GAMAL NO-3 (detergent), 0.4 g aseptina, 1.6 g ethanol, 0.04 g BRONOPOL (preservative), 6 g sodium chloride, 106 g of water.

### Example 16

A tooth paste comprising fulvic acid in an amount to obtain a final concentration of 1,000 to 20,000 mg/L, etheral oils or their compositions or else fruit essences in an amount of 1-10% by weight, glycerol in an amount of 5-10% by weight, herb extracts in an amount of 0.10-10% by weight and cleaning substances in an amount of 20-35% by weight dispersed in water in an amount of 45-60% by weight, and dyes and whitening components in an amount of 1-2% by weight.

Titanium dioxide may be used as a whitening component; sage leaves, camomile or marigold flowers extracts may be used as beneficial preferred herb extracts.

### Example 17

A bath salt preparation: 97 g salt (NaCl) containing occluded fulvic acid in an amount to obtain a final concentration of 1,000 to 20,000 mg/L and 3 g pine ethereal oil or ethereal oils composition is prepared.

### Example 18

A hair balm comprising fulvic acid in an amount to obtain a final concentration of 1,000 to 20,000 mg/L, herb extracts in an amount of 0.01-10% by weight, anti-electrostatic components in an amount of 3-4% by weight, components preventing excessive drying of hair and skin in an amount of up to 2% by weight, glycerol in an amount or 1-5% by weight, preservative and stabilizers in an amount of 0.05-0.50% by weight and water to 100% by weight.

As an anti-electrostatic component, the present balm contains an alcoholic solution of trimethylamine and ammonium chloride salt, obtained from fatty animal-derived amines; as thickening agent—acting also as stabilizing agent—cosmetic alcohol; as agent preventing excessive dryness of hair and skin—plant oils, acting simultaneously as co-emulsifying agents; and glycerol for ease in spreading and penetration of the balm, in particular of its fulvic acid and herb extracts. As an acidic environment stops multiplication of bacteria, the balm according to the invention contains citric acid or fumaric acid in an amount of 0.1% as well as a preservative known as BRONOPOL and fragrant compositions.

### Example 19

Cosmetic masks are well known cosmetic preparations serving many different purposes. A cosmetic mask comprising fulvic acid in an amount to obtain a final concentration of 1,000 to 20,000 mg/L, 20 g natural therapeutic mud, 10 g of humic acid, 10 g magnesium carbonate, 5 g zinc oxide, 0.2 g citiric acid, 5 g herb extract or powdered plant material, and q.s. distilled water is prepared.

### Example 20

A lotion or cream comprising fulvic acid in an amount to obtain a final concentration of 1,000 to 20,000 mg/L is prepared. For example

| Water | QS |
|---|---|
| Natural Oils | 1-15% |
| Emulsifiers | 1-10% |
| Emollients | 1-10% |
| Active Ingredients | 1-10% |
| Humectant | 1-5% |
| Fulvic Acid | 5,000 mg/L |
| Herbal Extracts | 0.01-5% |
| Aesthetic Enhancer | 0.25-2.5% |
| Fragrance | 0.25-2% |
| Viscosity and/or Rheology Modifiers | 0.025-1.5% |
| pH Adjustor | 0.01-1% |
| Vitamins | 0.01-1% |
| Preservatives | 0.1-1% |

### Example 21

A shampoo comprising fulvic acid in an amount to obtain a final concentration of 1,000 to 20,000 mg/L is prepared. For example

| Water | QS |
|---|---|
| Surfactants | 10-70% |
| Fulvic Acid | 5,000 mg/L |
| Conditioning Agents | 0.5-5% |
| Silicone | 0.25-5% |
| Humectants | 0.5-5% |
| Herbal Extracts | 0.01-5% |
| Viscosity and/or Rheology Modifiers | 0.025-3% |
| Fragrance | 0.25-2% |
| Sodium salt of EDTA | 0.05-1% |
| pH Adjustor | 0.01-1% |
| Vitamins | 0.01-1% |
| Preservatives | 0.1-1% |

### Example 22

A rinse off conditioner comprising fulvic acid in an amount to obtain a final concentration of 1,000 to 20,000 mg/L is prepared. For example

| Water | QS |
|---|---|
| Conditioning Agents | 1.0-15% |
| Emulsifiers | 1.0-10% |
| Fulvic Acid | 5,000 mg/L |
| Humectants | 0.5-5% |
| Herbal Extracts | 0.01-5% |
| Viscosity and/or Rheology Modifiers | 0.025-3% |
| Fragrance | 0.25-2% |
| Sodium salt of EDTA | 0.05-1% |
| pH Adjustor | 0.01-1% |
| Vitamins | 0.01-1% |
| Preservatives | 0.1-1% |

### Example 23

A leave in conditioner comprising fulvic acid in an amount to obtain a final concentration of 1,000 to 20,000 mg/L is prepared. For example

| Water | QS |
|---|---|
| Conditioning Agents | 1.0-10% |
| Emulsifiers | 1.0-10% |
| Herbal Extracts | 0.5-10% |
| Fulvic Acid | 5,000 mg/L |
| Viscosity and/or Rheology Modifiers | 0.025-3% |
| Mineral Salts | 0.1-2.5% |
| Vitamins | 0.1-2.5% |
| Fragrance | 0.25-2% |
| Amino Acids | 0.1-2% |
| pH Adjustor | 0.01-1% |
| Preservatives | 0.1-1% |

### Example 24

A moisturizing mist comprising fulvic acid in an amount to obtain a final concentration of 1,000 to 20,000 mg/L is prepared. For example

| Water | QS |
|---|---|
| Moisturizers | 1-20% |
| Emulsifiers | 0.5-5% |
| Fulvic Acid | 5,000 mg/L |
| Humectants | 0.5-5% |
| Herbal Extracts | 0.01-5% |
| Fragrance | 0.25-2% |
| Sodium salt of EDTA | 0.05-1% |
| pH Adjustor | 0.01-1% |
| Vitamins | 0.01-1% |
| Preservatives | 0.1-1% |

### Example 25

A shower gel comprising fulvic acid in an amount to obtain a final concentration of 1,000 to 20,000 mg/L is prepared. For example

| Water | QS |
|---|---|
| Surfactants | 10-70% |
| Fulvic Acid | 5,000 mg/L |
| Conditioning Agents | 0.5-5% |
| Silicone | 0.25-5% |
| Humectants | 0.5-5% |
| Herbal Extracts | 0.01-5% |
| Viscosity and/or Rheology Modifiers | 0.025-3% |
| Fragrance | 0.25-2% |
| Sodium salt of EDTA | 0.05-1% |
| pH Adjustor | 0.01-1% |
| Vitamins | 0.01-1% |
| Preservatives | 0.1-1% |

### Example 26

A facial mask comprising fulvic acid in an amount to obtain a final concentration of 1,000 to 20,000 mg/L is prepared. For example

| Water | QS |
|---|---|
| Natural Clays and Starches | 1.0-15% |
| Natural Oils | 1-10% |
| Emulsifiers | 1-10% |
| Emollients | 1-10% |
| Humectants | 1-5% |
| Fulvic Acid | 5,000 mg/L |
| Herbal Extracts | 0.01-5% |
| Aesthetic Enhancers | 0.25-2.5% |
| Fragrance | 0.25-2% |
| Viscosity and/or Rheology Modifiers | 0.025-1.5% |
| pH Adjustor | 0.01-1% |
| Vitamins | 0.01-1% |
| Preservatives | 0.1-1% |
| Pigment | 0.01-1% |

### Example 27

A hair spray comprising fulvic acid in an amount to obtain a final concentration of 1,000 to 20,000 mg/L is prepared. For example

| Water | QS |
|---|---|
| Alcohol | 40-60% |
| Fulvic Acid | 5,000 mg/L |
| Conditioning Agents | 0.5-5% |
| Silicone | 0.25-5% |
| Humectants | 0.5-5% |
| Herbal Extracts | 0.01-5% |
| pH Adjustor/Neutralizing Agent | 0.25-3% |
| Fragrance | 0.25-2% |
| Vitamins | 0.01-1% |
| Preservatives | 0.1-1% |

### Example 28

A liquid nutritional supplement comprising fulvic acid in an amount to obtain a final concentration of 1,000 to 20,000 mg/L is prepared. For example

| Water | QS |
|---|---|
| Colloidal Minerals | 0.5-5% |
| Fulvic Acid | 5,000 mg/L |
| Sweeteners | 2-10% |
| Flavorings | 0.25-1.5% |
| Preservatives | 0.1-1% |
| pH Adjustor/Neutralizing Agent | 0.25-3% |

### Example 29

A liquid nutritional supplement comprising fulvic acid in an amount to obtain a final concentration of 1,000 to 20,000 mg/L is prepared. For example

| Water | QS |
|---|---|
| Sweeteners | 2-10% |
| Fulvic Acid | 5,000 mg/L |
| Vitamins | 0.0001-0.01% |
| Flavorings | 0.25-1.5% |
| Herbal Extracts | 0.01-5% |
| pH Adjustor/Neutralizing Agent | 0.25-3% |
| Preservatives | 0.1-1% |

### Example 30

A liquid nutritional supplement comprising fulvic acid in an amount to obtain a final concentration of 1,000 to 20,000 mg/L is prepared. For example

| Water | QS |
|---|---|
| Sweeteners | 2-10% |
| Fulvic Acid | 5,000 mg/L |
| Amino Acids | 0.001-.05% |
| Flavorings | 0.25-1.5% |
| Herbal Extracts | 0.01-5% |
| pH Adjustor/Neutralizing Agent | 0.25-3% |
| Preservatives | 0.1-1% |

### Example 31

A liquid nutritional supplement comprising fulvic acid in an amount to obtain a final concentration of 1,000 to 20,000 mg/L is prepared. For example

| Water | QS |
|---|---|
| Sweeteners | 2-10% |
| Fulvic Acid | 5,000 mg/L |
| Noni | 01-10% |
| Flavorings | 0.25-1.5% |
| Preservatives | 0.1-1% |

### Example 32

A liquid nutritional supplement comprising fulvic acid in an amount to obtain a final concentration of 1,000 to 20,000 mg/L is prepared. Examples of other ingredients present in the nutritional supplement include, but are not limited to, water, sweeteners, juices, flavorings, minerals, trace elements, amino acids, vitamins, plants, plant extracts, plant derived materials, colorings, preservatives, texturizers, and pH adjustors.

Further examples of liquid nutritional supplements include, but are not limited to fulvic acid in an amount to obtain a final concentration of 1,000 to 20,000 mg/L in combination with the proprietary formulas of the products ELITE Mineral Star, ELITE Cartilage Star, ELITE MULTI-STAR, Nutraceutica Reflex-3, Nutraceutica Lemon Minerals, Nutraceutica Raspberry Minerals, Nutraceutica Mega Vitamins with Rare Earth Minerals, GNC Liquid Multi-Colloidal Minerals, NBTY/Vitamin World Tongan Limu Moui, and Now Foods Colloidal Mineral, each available from Advantage Marketing, Inc.

### Example 33

The subjects were twin 45 year old males who historically had suffered from male pattern baldness for a period of ten years. The test subject was treated three times daily with an oral solution comprising 10 mg fulvic acid three times a day for a period of just over three months. At the end of four weeks there was no observable change; however, by the 12th week there was a definite subjective increase in head hair in comparison to the twin.

### Example 34

Thirty subjects having male pattern baldness are identified for study. Fifteen of the subjects are treated orally with 30 mg of fulvic acid per day while the remaining 15 are given a placebo. After 12 weeks there is a definite subjective increase in hair growth on the scalp. After 20 weeks, both subjective and objective increases in hair growth on the scalp are observed.

While this invention has been described in certain embodiments, the present invention can be further modified within the spirit and scope of this disclosure. This application is therefore intended to cover any variations, uses, or adaptations of the invention using its general principles. Further, this application is intended to cover such departures from the present disclosure as come within known or customary practice in the art to which this invention pertains and which fall within the limits of the appended claims.

### REFERENCES

Baj Z. 1993. Effect of Tolpa Peat Preparation on Selected Immune Parameters in Healthy Volunteers. Acta. Pol. Pharm. 50:481-9.
Banaszkiewicz W. and M. Drobnik. 1994. The influence of natural peat and isolated humic acid solution on certain indices of metabolism and of acid-base equilibrium in experimental animals. Rocz. Panstw. Zakl. Hig. 45(4):353-60.
Beer A. M., J. Lukanov and P. Sagorchev. 2000. The influence of fulvic and ulmic acids from peat, on the spontaneous contractile activity of smooth muscles. Phytomedicine October. 7(5):407-15.
Beer A. M., H. E. Junginger, J. Lukanov and P. Sagorchev. 2003. Evaluation of the permeation of peat substances through human skin in vitro. Int. J. Pharm. 253:169-175.
Bhattacharya S. K. 1995. Activity of shilajit on alloxan-induced hyperglycemia in rats. Fitoterapia Volume LXVI, No. 4, 1995. p. 328.
Chen Y. and T. Aviad. 1990. Effects of Humic Substances on Plant Growth. Chapter 7 in: MacCarthy, et. al., Humic Substances in Soil and Crop Sciences: Selected Readings. American Soc. of Agronomy, Madison, Wis.
Chen Y., C. E. Clapp, H. Magen, and V. W. Cline. 1999. Stimulation of plant growth by humic substances: Effects on iron availability. p. 255-263. In E. A. Ghabbour and G. Davies (eds.) Understanding Humic Substances: Advanced Methods, Properties and Applications. The Royal Society of Chemistry, Cambridge.
Covington B. R., W. S. Ramsey, L. W. Greene and F. M. Byers. 1997. Effects of humate on feedlot performance and carcass characteristics in feedlot lambs. J. Anim. Sci. 75(Supp.1):270.
Day K. S., R. Thornton and H. Kreeft. 2000. Humic acid products for improved phosphorus fertilizer management. pp. 321-326 in Humic Substances—Versatile Components of Plants, Soil and Water. E. A. Ghabbour and G. Davies (eds.) Royal Society of Chemistry, Cambridge, UK.
Dekker J. and C. E. Elizabeth, 2001. Fulvic acid and its use in the treatment of various conditions. US Patent Submission 807004.
Fraioli A., S. De Angelis Curtis, G. Ricciuti, A. Serio and G. D'Ascenzo. 2001. Effect of water of Anticolana Valley on urinary sediment of renal stone formers. Clin. Ter. 152(6):347-51.
Freeman P. G., 1969. The use of lignite products as plant growth stimulants. Bureau of Mines Circular 8471, United States Department of Interior. pp. 150-153, 160, 162, 164.
Ghabbour E. and G. Davies. 1994. Isolation of humic substances from aquatic and terrestrial plants. J. Appl. Phycol. 6:459.
Ghosal S. 2003. Delivery system for pharmaceutical, nutritional and cosmetic ingredients. May 6, 2003. U.S. Pat. No. 6,558,712.
Grassi M., M. C. Lucchetta, G. B. Rini and S. Raffa. 2003. Fangotherapy in chronic degenerative rheumatic diseases. Clin. Ter. 2003 January-Feburary 154(1):45-8.
Insam H., 1996. Microorganisms and humus in soils. Chapter 6 in: Piccolo, A., 1996. Humic Substances in Terrestrial Ecosystems, Elsevier, Amsterdam.
Lotosh T. D. 1991. Experimental bases and prospects for the use of humic acid preparations from peat in medicine and agricultural production. Nauchnye Doki Vyss Shkoly Biol Nauki 10:99-103.
Madej J. A., J. Kuryszko and T. Garbulinski. 1993. Influence of Long-Term Administration of Tolpa Peat Preparation on Immune Reactivity in Mice. I. Morphological Changes in Thymus. Acta. Pol. Pharm. 50:397-404.
Madej J. A., J. Kuryszko and T. Garbulinski. 1993. Influence of Long-Term Administration of Tolpa Peat Preparation on Immune Reactivity in Mice. 2. The Effect of Intermittent TPP administration on the morphological picture of lymphatic organs. Acta. Pol. Pharm. 50:405-408.
Madej J. A., J. Kuryszko and T. Garbulinski. 1993. Influence of Long-Term Administration of Tolpa Peat Preparation on Immune Reactivity in Mice. 3. The effect on primary humoral response to sheep erythrocytes. Acta. Pol. Pharm. 50:491-496.
Nardi S., 1996, Biological Activity of Humus. Chapter 9 in: A. Piccolo (Ed.) Humic Substances in Terrestrial Ecosystems, Elsevier, Amsterdam.
Piccolo A., 1996. Humus and soil conservation. Chapter 5 in: Humic Substances in Terrestrial Ecosystems, A. Piccolo (Ed.) Elsevier, Amsterdam.
Stevenson F. J., 1994. Humus Chemistry—Genesis, composition, reactions, 2nd Ed.: New York, John Wiley and Sons, p. 307-317.
Tolpa S., T. Gersz, S. Ritter, R. Kukla, M. Skrzyszewska and S. Tomkov. 1998. Peat-derived bioactive products, pharmaceutical and cosmetic compositions containing said products and processes for producing said products and compositions. U.S. Pat. No. 5,747,050.
University of California, Berkely Wellness Letter. Dead doctors don't lie, but people who sell colloidal minerals . . . . June, 1997 Edition.
Vaughan D. and R. E. Malcolm, (Eds.) 1985. Soil Organic Matter and Biological Activity. Martinus Njihoff/Dr. W. Junk Publishers, Dordrecht.
Visser S. A. 1987. Effect of humic substances on mitochondrial respiration and oxidative phosphorylation. Science of the Total Environment 62:347-354.

1. A method of promoting hair growth in a subject, the method comprising:
delivering to the subject at least 5 mg of fulvic acid per day.

2. The method according claim 1, wherein the fulvic acid is purified or substantially purified.

3. The method according to claim 2, wherein the fulvic acid is purified or substantially purified using a XAD-8.

4. The method according to claim 2, wherein the fulvic acid is purified or substantially purified from an aquatic source.

5. The method according to claim 2, wherein the fulvic acid is not purified or substantially purified from coal, shilajit, or peat.

6. The method according to claim 1, wherein the fulvic acid is not oxifulvic acid.

7. The method according to claim 1, wherein from 20 to 30 mg of fulvic acid is delivered to the subject per day.

8. The method according to claim 1, wherein 25 mg of fulvic acid is delivered to the subject per day.

9. A composition for human use, the composition comprising at least 700 mg/L fulvic acid.

10. The composition of claim 9, wherein the composition is in a form selected from the group consisting of tablets, capsules, granules, sprays, inhalers, suppositories, sprays, patches, balms, drops, cosmetics, supplements, foods, creams, ointments, liquids, drinks, beverages, lotions, milky lotions, facial packs, bathing agents, bath detergents, facial cleansing agents, shaving creams, hair lotions, hair-care compositions, and shampooing agents.

11. The composition of claim 9, wherein the composition further comprises a carrier or an adjuvant.

12. The composition of claim 9, wherein the composition comprises from 700 to 20,000 mg/L fulvic acid.

13. The composition of claim 9, wherein the composition comprises from 700 to 1000 mg/L fulvic acid.

14. The composition of claim 9, wherein the composition comprises 3000 mg/L fulvic acid.

15. The composition of claim 9, wherein the fulvic acid is purified or substantially purified.

16. The composition of claim 9, wherein the fulvic acid is purified or substantially purified using XAD-8.

17. The composition of claim 16, wherein the fulvic acid is purified or substantially purified from an aquatic source.

18. The composition of claim 16, wherein the fulvic acid is not purified or substantially purified from coal, shilajit, or peat.

19. The composition of claim 9, wherein the fulvic acid is not oxifulvic acid.
